(19) United States
(12) Patent Application Publication
(10) Pub. No.: US 2015/0087952 A1
(43) Pub. Date: Mar. 26, 2015

(54) SMARTPHONE AND ECG DEVICE MICROBIAL SHIELD

(71) Applicant: AliveCor, Inc., San Francisco, CA (US)

(72) Inventors: David E. ALBERT, San Francisco, CA (US); Christopher Ammon MYLES, San Francisco, CA (US)

(21) Appl. No.: 14/494,191

(22) Filed: Sep. 23, 2014

Related U.S. Application Data
(60) Provisional application No. 61/881,593, filed on Sep. 24, 2013.

(51) Int. Cl. A61B 19/02; A61B 5/044; A61B 5/00; A61B 5/0408; A61B 5/0404

(52) U.S. Cl. CPC A61B 19/02; A61B 5/04085; A61B 5/0404; A61B 5/6898; A61B 5/6823; A61B 5/044; A61B 5/6824; A61B 5/6828; A61B 2019/0201
USPC 600/384; 600/393; 600/382

(57) ABSTRACT

A shield can inhibit transfer of microbes and bodily fluids from a first side to a second side of the shield, while allowing an ECG device adjacent the first side of the shield to sense separate electrical potentials on skin adjacent the second side. The shield comprises a flexible sheet with a first electrically conductive portion and a second electrically conductive portion; the first and second electrically conductive portions are separated from one another by an electrically insulating portion.

### CROSS-REFERENCE

This application claims the benefit of U.S. Provisional Application No. 61/881,593, filed Sep. 24, 2013, which application is incorporated herein by reference.

### BACKGROUND

A number of arrangements are available for protecting medical monitoring devices and probes from contamination during use. For example, disposable sheaths are available for thermometers and protective covers are available for stethoscope heads.

A shield for an ECG monitoring device can inhibit the transfer of microbes and bodily fluids across the shield while allowing the ECG device to sense separate electrical potentials on the skin of a subject or multiple subjects.

### SUMMARY

An aspect of the present disclosure relates to a microbial shield for inhibiting transfer of microbes and bodily fluids from a first side to a second side of the shield, while allowing an ECG device adjacent the first side of the microbial shield to sense separate electrical potentials on a skin surface that is adjacent to the second side of the microbial shield.

The microbial shield can comprise a flexible sheet with a first electrically conductive portion and a second electrically conductive portion, the first and second electrically conductive portions separated from one another by an electrically insulating portion to allow functioning of the ECG device.

In an aspect of the present disclosure, an ECG monitoring system comprises a handheld ECG sensing device which comprises two or more ECG electrodes or sensors, and a disposable shield in communication with the two or more ECG electrodes.

The disposable shield comprises a first electrically conductive portion, a second electrically conductive portion, and an insulating portion separating the first and the second electrically conducting portions, and wherein the ECG sensing device senses separate electrical potentials on a first and a second skin segment of a subject when the first electrically conductive portion and the second electrically conductive portion are contacted by the first and the second skin segments of a subject.

The disposable shield can comprise an envelope.

A skin segment of the subject can for example comprise a skin segment of a chest of the subject or a skin segment of a limb of a subject. The handheld ECG sensing device can comprise a smartphone or other mobile computing device.

Another aspect of the present disclosure describes a method for managing contamination of a sensing device comprising providing a handheld ECG sensing device comprising two or more ECG electrodes or sensors, providing a shield between a skin surface of a subject and the ECG sensing device, wherein the ECG sensing device senses separate electrical potentials on a first and a second skin segment of a subject without the first and the second skin segments of the subject contacting the ECG sensing device, sensing an ECG from the subject, and disposing of the shield. The disposable shield can comprise an envelope. A skin segment of the subject can for example comprise a skin segment of a chest of the subject or a skin segment of a limb of a subject. The handheld ECG sensing device can comprise a smartphone or other mobile computing device. The shield can comprise a first and a second electrically conductive portion and an insulating portion separating said first and said second electrically conducting portions.

The handheld ECG sensing device can comprise a display, which can display the sensed ECG from the subject on the ECG sensing device.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features described herein are set forth with particularity in the appended claims. A better understanding of the features and advantages of the present disclosure will be obtained by reference to the following detailed description that sets forth illustrative examples, and the accompanying drawings.

Like reference numerals in the figures represent and refer to the same or similar element or function. Implementations of the disclosure may be better understood when consideration is given to the following detailed description thereof. Such description makes reference to the annexed pictorial illustrations, schematics, graphs, and drawings. The figures are not necessarily to scale and certain features and certain views of the figures may be shown exaggerated, to scale, or in schematic in the interest of clarity and conciseness. In the drawings:

FIG. 1 shows the steps of a method for managing contamination of a sensing device

FIG. 2 shows a microbe shield with a handheld ECG sensing device.

FIG. 3 shows another aspect of a microbe shield.

FIG. 4 shows an edge view of a microbe shield.

FIG. 5A shows a top view of a microbe shield.

FIG. 5B shows a bottom view of a microbe shield.

FIG. 6 shows a user inserting a smart phone into a microbe shield envelope.

### DETAILED DESCRIPTION

It is to be understood that the disclosed subject matter is not limited in its application to the details of construction, experiments, exemplary data, and/or the arrangement of the components set forth in the following description, or illustrated in the drawings. The presently disclosed subject matter is capable of other embodiments or of being practiced or carried out in various ways. Also, it is to be understood that the phraseology and terminology employed herein is for purpose of description only and should not be regarded as limiting in any way.

In the following detailed description numerous specific details are set forth in order to provide a more thorough understanding. However, it will be apparent to one of ordinary skill in the art that the subject matter within the disclosure may be practiced without these specific details. In other instances, well-known features have not been described in detail to avoid unnecessarily complicating the instant disclosure.

Further, unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the inventive concept. This description should be read to include one or at least one and the singular also includes the plural unless it is obvious that it is meant otherwise.

A microbial shield is described herein that is configured to be used with a portable or handheld ECG device such as that disclosed in U.S. Pat. No. 8,301,232 and U.S. Pat. No. 8,509,882, the contents of each are incorporated herein by reference. The U.S. Pat. No. 8,301,232 and U.S. Pat. No. 8,509,882 patents disclose a portable or handheld ECG device that can communicate with a smartphone or other mobile computing device. In one variation, a smartphone protective case incorporates an ECG sensing device.

The ECG sensing device uses an electrode or sensor assembly configured to sense heart-related signals upon contact with a user's skin, and converts the sensed heart-related signals to ECG electrical signals. The electrode or sensor assembly is positioned on an outer surface of the ECG sensing device or, in one variation, on the outer surface of the smartphone protective case. A converter transmits the ECG electrical signals, which are received by a computer or smartphone. It is anticipated that health care professionals including nurses will use such devices to measure, transmit and record a patient's ECG since the ECG sensing device allows rapid monitoring of multiple patients.

When applying the handheld ECG sensing device to patients, a microbial shield can for example prevent the spread of disease from one patient to another or for example transmission of microbes from a patient to him or herself. If a handheld ECG sensing device is incorporated with a smartphone, then additional shielding for the smartphone can be provided to protect the smartphone and ECG sensing device from contamination.

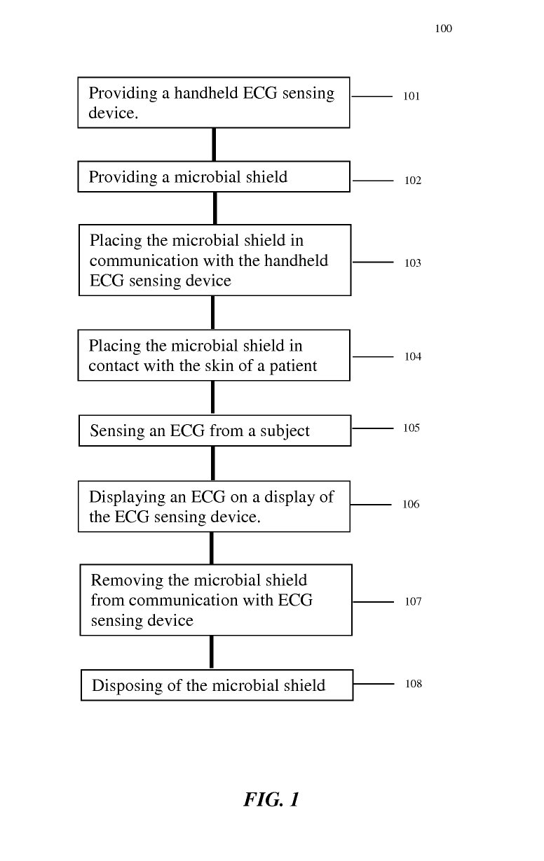

Referring now to the drawings, FIG. 1 shows the exemplary steps of a method 100 for managing contamination of a sensing device. A user such for example a nurse, doctor, health care provider, or subject can be provided with a handheld ECG sensing device in a step 101 that can for example couple with a smartphone case or any other similar mobile computing device. The ECG sensing device can comprise two or more sensors for sensing an electrical potential on the skin surface of a subject. For example, the sensors could sense a signal from the skin surface of a subject when the subject's skin touches the sensors. For example, in an ECG sensing device with two sensors, a subject can touch one sensor with a finger from his right hand and the second sensor with a finger from his left hand. It is understood that there are other skin segments of a subject that are usable with an ECG sensing device, including the chest of a subject and a limb of a subject. The sensors of the ECG sensing device can sense an electrical potential on the surface of a skin segment of the chest of a subject when one or multiple sensors of the ECG sensing device are in contact with a segment or segments of the subject's chest. Likewise, an electrical potential on the surface of a skin segment of a limb of a subject can be measured when a sensor or sensors of the ECG sensing device are in contact with an arm or leg of a subject. Because pathogenic microbes can reside on the skin surface of a subject, direct contact of a skin segment of a subject with the ECG sensing device can result in the transfer of a pathogenic microbe onto the ECG sensing device. This transfer of microbes to the ECG sensing device is of particular concern in for example, a hospital or other clinical setting where one ECG sensing device might be used on multiple subjects. Subjects in hospitals and other clinical settings tend to carry infection causing pathogenic microbes on their skin. If there is direct contact of the ECG sensing device with the skin of a first subject having a pathogenic microbe on their skin followed by direct contact of that same ECG sensing device with a second subject, the pathogenic microbes on the skin of the first subject can transfer to the ECG sensing device and then from the ECG sensing device to the second subject. Similarly, pathogenic microbes can pass in this way from an ECG sensing device to a nurse, doctor, or other health care provider.

A user can further be provided with a microbial shield in a step 102. The microbial shield prevents a transfer of microbes from a subject the ECG sensing device by preventing direct contact of the skin of a subject with the ECG sensing device. The microbial shield can comprise two or more electrically conductive portions and an insulating portion situated between the electrically conducting portions. The two or more electrically conductive portions can comprise any conductive metal or metal foil including for example copper, aluminum, copper alloy, gold, and silver. The two or more electrically conductive portions can also comprise for example conductive fabrics which are available with, for example, semi-metallized and metal conductive yarns. It is understood that any conductive material can be suitable. The insulating material is any material that serves as an insulator such as for example a plastic, silicone, or other polymer or polymer blend material. Likewise a paper or cotton based material can be used as insulating portion as well. The insulating portion is positioned between the two electrically conducting portions so that that they are for example separated from each other and an electrical signal cannot pass from one electrically conducting portion to another. The insulating portion can for example have a larger overall surface than the electrically conducting portion and thus can extend outwards to provide a large shield surface. The two or more electrically conducting portions are positioned on the microbial shield so that they can communicate an electric signal to the two or more sensors. The microbial shield can be configured as a sheet or as an envelope. The microbial shield has two surfaces. When used together with an ECG sensing device, one surface of the microbial shield is an inward surface which is oriented towards the ECG sensor and communicates with the ECG sensing device sensors. The second surface of the microbial shield is oriented outwards towards the environment when used with an ECG sensing device. The outward surface of the microbial shield can be contacted by a subject. The two or more conducting portions of the microbial shield which are part of the microbial shield also have an inward surface and an outward surface. An electrical signal can be conducted by the electrically conducting portions from their outward surface which faces the environment towards their inward surfaces which is oriented towards the ECG sensing device. Thus for example, an electrical signal from the skin surface of a subject can be conducted through the microbial shield from the outward surface of the microbial shield to the inward surface when the outward surface of the two or more conducting portions is contacted by a skin surface of a subject.

In a step 103 a user places the microbial shield in communication with the ECG sensing device. For example, the user can place the microbial shield in contact with the ECG sensing device by positioning the inward side of the microbial shield against the ECG sensing device so that for example the inward surface of the two or more electrically conducting portions of the microbial shield contact the two or more sensors of the ECG sensing device. The two or more conducting portions of the microbial shield communicate with the two or more ECG device sensors in a one to one fashion. It is understood that that the two or more conducting portions of the microbial shield can communicate with the two or more sensors of the ECG sensing device by direct surface to surface contact, by a capacitive connection, or any other method of connecting conductors that are known in the art. The microbial shield can have at least one adhesive surface that adheres the inward surface of the microbial shield to the ECG sensing device. Alternatively, the microbial shield can have an adhesive surface on its outward facing surface to adhere to a skin surface of a subject. If the microbial shield is the envelope variation, the user places the ECG sensing device along with any mobile computing device that the ECG sensing device is coupled to inside of the envelope. Alternatively, the user places only the ECG sensing device in the envelope and can then couple the ECG and microbial shield envelope to a mobile computing device. In the envelope variation of the microbial shield, the user places the electrically conducting portions of the microbial sensing device in communication with the sensors of the ECG sensing device as the user would in the sheet variation of the microbial shield. The ECG sensing device is positioned relative to the microbial shield so that microbial shield prevents contact of the ECG sensing device with contaminants associated with the subject or any other contaminants in the environment. If the ECG sensing device is coupled with a mobile computing device, the microbial shield can prevent the mobile computing device from being contacted by contaminants associated with the subject or any other contaminant in the environment. For example, in the variation where the microbial shield is a sheet, a user can place a microbial shield that for example has larger dimensions than the ECG sensing device or larger dimensions than the ECG sensing device in combination with a mobile computing device against the ECG sensing device or the ECG sensing device and mobile computing device together. In this way an entire surface of the ECG sensing device or the ECG sensing device together with a mobile computing device is covered by the microbial shield sheet. Alternatively, the outward surface of the sheet variation of the microbial shield can first be placed against a subject such as for example against the chest of a subject, and then the ECG sensing device or ECG sensing device together with the mobile computing device can be placed against the inward surface of microbial shield so that the ECG sensing device or the ECG sensing device together with the mobile computing device does not come into contact with the chest of the subject. In the variation where the microbial shield is an envelope, the user can place the ECG sensing device or the ECG sensing device and a mobile computing device entirely inside the envelope so that the ECG sensing device or the ECG sensing device together with a mobile computing device are substantially covered. Further, the microbial envelope with the ECG sensing device or the ECG sensing device together with a mobile computing device can be sealed inside the envelope either permanently as for example with an adhesive or reversibly as with for example a zipper or interlocking mechanism.

In a step 104, a user places the outward surface of the microbial shield in contact with a skin surface segment of a subject. The outward surface of the microbial shield can be placed on any skin surface of the subject that is suitable for recording an ECG signal. For example, the microbial shield can be placed on a subject's chest so that the electrically conductive portions of the microbial shield are in contact with the subject's skin over an area of the subject's chest where an ECG signal might be recorded. Alternatively, the subject can be provided with an ECG sensing device coupled with a microbial shield, and the subject's fingers can be brought into contact with the electrically conductive portion of the microbial shield by the subject himself or the user can place the subject's fingers in contact with the electrically conductive portions. For example the microbial shield can be placed over the left chest of the subject where the heart is located and where traditional ECG leads are typically placed. It is important to note, that step 104 can occur either before step 103 or after step 103. Step 103, at least in part describes placing the microbial shield in communication with an ECG sensing device, and step 104, at least in part describes placing the outward surface of the microbial shield in contact with a skin surface of a subject. If a user should choose, the microbial shield can be for example first placed against or adhered to a surface of the ECG sensing device with the inward surface of the microbial shield contacting the ECG sensing device, and then the ECG sensing device surface that is covered by the microbial shield can be placed into contact with a skin surface of a subject. Alternatively, if a user should choose, a microbial shield can for example first be placed against or adhered to a skin surface of a subject such as for example the chest of a subject, and then an ECG sensing device can be placed in communication with the microbial shield that is in contact with the subject's chest. Whether step 104 precedes step 103 or follows step 103, the ECG sensing device or the ECG sensing device and mobile computing device do not contact the skin surface of the subject, because the microbial shield serves as a barrier.

In a step 105, an ECG is sensed from a subject. The two ECG sensors on the ECG sensing device can sense an ECG when the skin of a subject contacts the electrically conductive portions of the microbial shield that communicate with the two sensors. For example, a subject can contact a first electrically conductive portion of a microbial shield with their right thumb and a second electrically conductive portion of a microbial shield with their left thumb. A signal is conducted by each respective conductive portion to a corresponding communicating sensor on the ECG sensing device so, for example, an electrical potential on the skin surface of the right and left thumbs of the subject can be measured by the ECG sensing device. The insulating portion of the microbial shield prevents any crossover of signal from one electrically conductive portion to another electrically conductive portion. The ECG sensing device senses an ECG via for example the apparatuses and methods described in U.S. Pat. No. 8,301,232 and U.S. Pat. No. 8,509,882.

In a step 106, a sensed ECG of a subject is displayed on a display screen of for example a mobile computing device that is coupled to the ECG sensing device. The display of the ECG on the screen of a mobile computing device can for example comprise a two lead ECG, three lead ECG, four lead ECG, six lead ECG, or 12 lead ECG. All leads can for example be displayed on the screen at one time, in groups, or separately.

In a step 107, the microbial shield can be removed from contact with the subject, and in a step 108 the microbial shield that is now contaminated due to contact with a subject is disposed of. A new clean or sterile microbial shield can be obtained by the user and used in substantially the same way outlined in the steps of the method 100 on a different subject or the same subject.

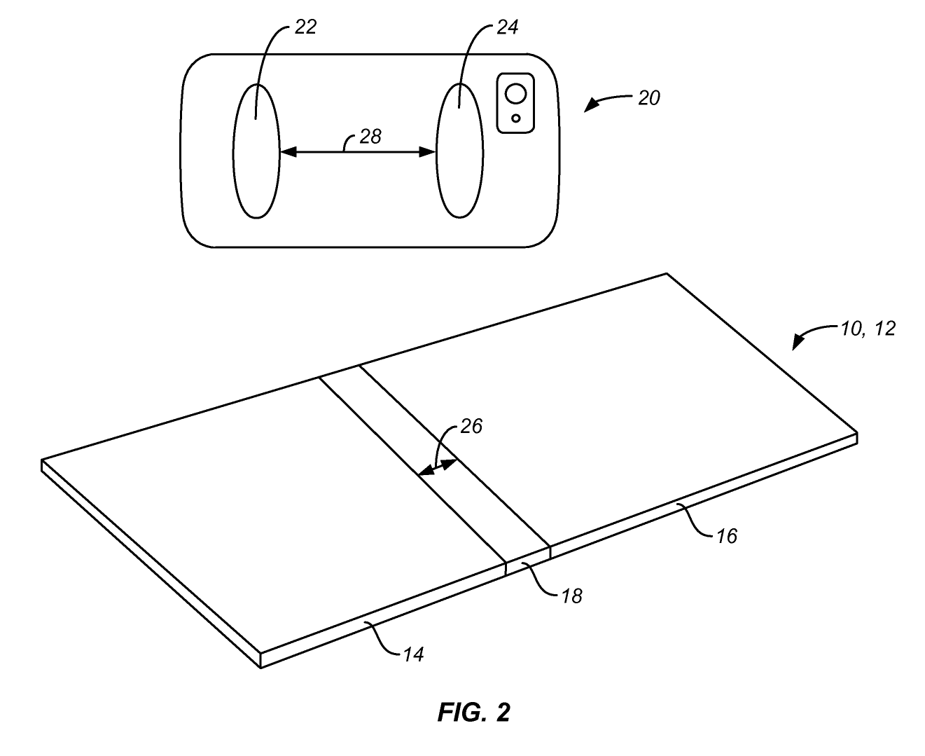

FIG. 2 shows an exemplary embodiment of a microbial shield 10. The microbial shield 10 comprises a sheet 12 having a first electrically conductive portion 14 and a second electrically conductive portion 16, the first and second electrically conductive portions 14 and 16, respectively, separated from one another by an electrically insulating portion 18. Also shown is a smartphone with a protective cover 20 incorporating an ECG sensing device having a first sensor 30 and a second sensor 24. The microbial shield 10 is proportioned to be larger than the smartphone with a distance 26 between the first and second electrically conductive portions 14 and 16, respectively, that is equal to or less than the distance 28 between the first and second sensors 22 and 24, respectively. This allows a user to place the microbial shield 10 on, for example, a patient's chest and place the smartphone with the protective cover 20 onto the microbial shield 10 such that the first sensor 22 contacts the first electrically conductive portion 14 of the microbial shield 10, and the second sensor 24 contacts the second electrically conductive portion 16 of the microbial shield 10. While the sheet 12 is shown as a rectangle with large first and second conductive portions, it is understood that other shapes and sizes are useable and that the size and shape of the first and second electrically conductive portions 14 and 16, respectively can vary.

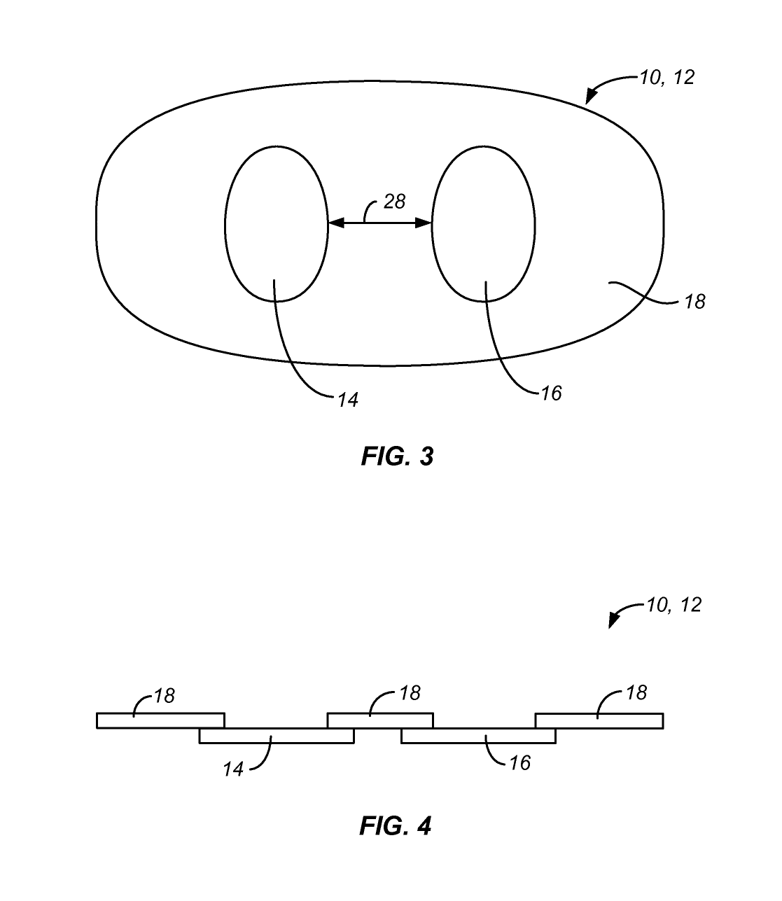

FIG. 3 shows an embodiment of the microbial shield 10 wherein the electrically insulating portion 18 comprises most of the sheet 12, and the first and second electrically conductive portions 14 and 16, respectively, are similar in size and shape to the first and second sensors 22 and 24, respectively, on the smartphone with the protective cover 20. The actual design can vary and will be influenced by manufacturing cost and ease of use.

The first and second electrically conductive portions can be constructed of a metal foil, a woven or non-woven conductive fabric, or the like. Non-limiting examples of suitable metal foils include copper, aluminum, copper alloy, gold, and silver foils. Suitable conductive fabrics are available with, for example, semi-metallized and metal conductive yarns.

Any electrically insulating material can be utilized for the electrically insulating portion 18. In some embodiments the electrically insulating material is flexible and can be silicone, a polymer such as polyethylene, or the like. Plastics, papers, and fabrics with suitable insulating properties can be used for the insulating portion 18 as well.

A suitable adhesive can be used to bond the edges of the first and second electrically conductive portions to the appropriate edges of the electrically insulating portion. Adhesive can be placed on a single side of the sheet variation of the microbial shield on one side of the sheet or both sides of the sheet. The type of adhesive used can vary depending on for example whether the adhesive is intended to contact the skin of a subject or a surface of an ECG sensing device or an ECG sensing device and a mobile computing device. Such methods for providing adhesion are known to those skilled in the art. An adhesive can be provided for any surface of the envelope variation of the microbial shield as well in the same manner.

The adhesive surface can be initially covered with a piece of paper or waxy paper and removed by the user immediately before use, much like the paper covering the adhesive portion of a band aid or adhesive dressing. The peel-off cover functions to maintain sterility prior to use of the microbial shield 10 on the side to be place on the patient.

Multiple microbial shields 10 can be provided on a roll of peel-off material. The multiple microbial shields 10 on a roll can be separable by perforations between the shields.

In another version, a microbial shield 10 comprises two peel-off covers similar to bandage material available commercially, thereby providing sterile surfaces to the entire microbial shield. Similarly, commercial procedures for providing peel-off covers for thermometer probe covers and adhesive bandages can be readily adapted to provide a sterile package for the microbial shield 10.

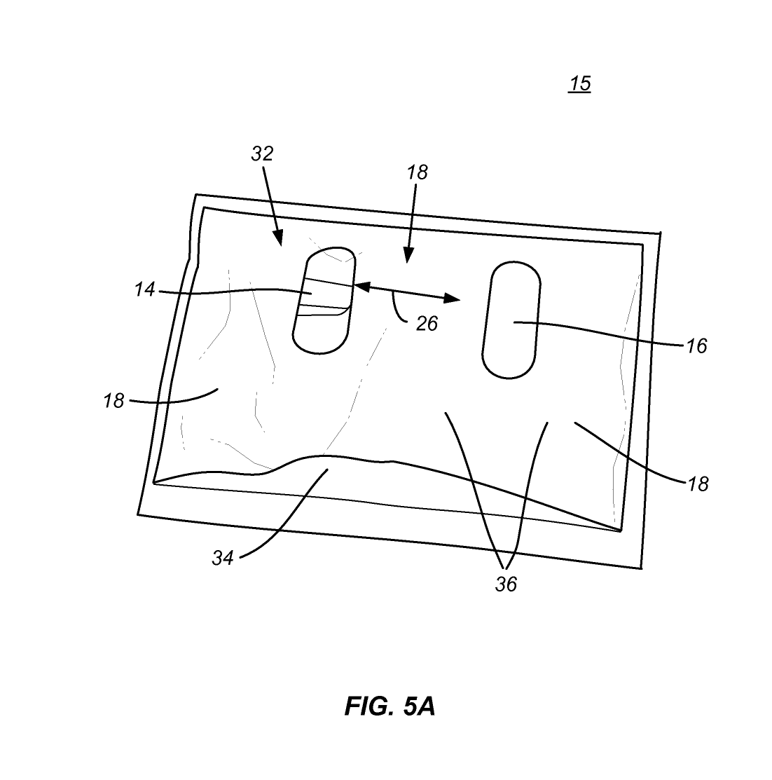

FIG. 4 shows an edge view of a variation of a microbial shield 10 comprising a sheet 12. Insulating portion 18, is positioned lateral to as well as in between conductive portions 14 and 16, so that each conductive portions 14 and 16 is surrounded by insulating portion 18. The insulating portion 18 that is between conductive portions 14 and 16 is positioned in the space 26 between electrically conductive portions 14 and 16.

The variation of the microbial shield shown in FIG. 4 also shows one way in which the microbial shield can be configured. In this variation of the microbial shield, there are two empty spaces in the insulating portion 18 which can comprise for example oval or circular holes or polygonal empty spaces 30 within the insulating portion 18. The electrically conductive portions 14 and 16 are positioned within these empty spaces 30 within the insulation portions 18 so that the electrical conducting portions 14 and 16 have a surface exposed on the outward surface of the microbial shield that faces the subject and the environment and the inward surface that faces the ECG sensing device. In this variation of the microbial shield, shown in FIG. 4, the electrically conductive portions 14 and 16 are sized to extend beyond the empty space 30 along the entire border of the empty space. The area of the electrically conductive portions 14 and 16 that extend beyond the empty space 30 contact the insulating portion 18 along the border of the empty space 30 forming an overlapping lip 32. The electrically conductive portions 14 and 16 can be sealed with the insulating portion 18 along the overlapping lip 32 so that the microbial shield is entirely sealed and impervious to penetration by microbial contaminants.

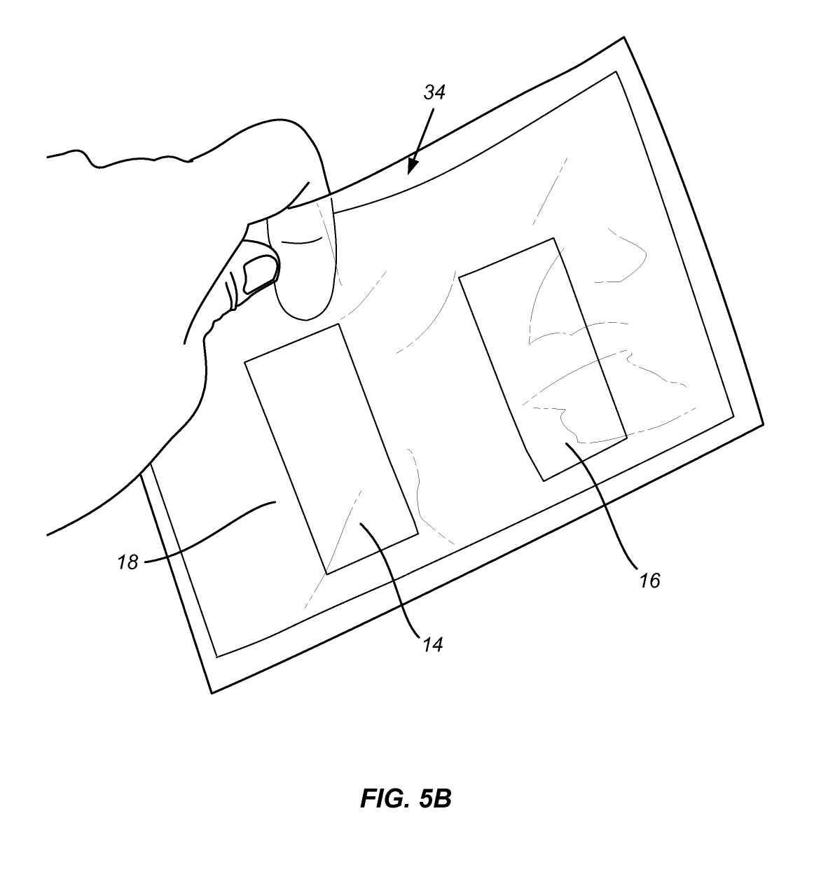

FIG. 5A and FIG. 5B show a version of the microbial shield 10 which is shaped as a bag or envelope 15. This version of the microbial shield can either remain open while used or it can be sealed. Similar to the sheet variation, the envelope comprises two or more electrically conductive portions 14 and 16, which can be contacted by the skin of a subject on the outside surface of the envelope 36. The envelope further comprises an insulating portion 18 which is positioned lateral to and in between the conductive portions 14 and 16. Overlapping lip 32 can be seen through the transparent outside surface of the envelope 36.

The envelope 15 of FIG. 5A and FIG. 5B can be configured so that only one wall of the envelope 15 comprises the conducting portions 14 and 16 and the remaining wall of the envelope can comprise entirely of the insulating portion 18. The area of the insulating portion 18 that is positioned between conducting portions 14 and 16 is in space 26. An ECG sensing device or an ECG sensing device together with a mobile computing device together can be placed inside the envelope through opening 34. Opening 34 can be sealable or non-sealable.

The sealable variation of envelope 15 can for example provide greater coverage of the device or devices within the envelope. A seal mechanism can comprise an adhesive on the interior surface of opening 34. The adhesive can be covered by a strip of paper that is removed by the user once the device or devices are placed inside thereby exposing the adhesive. Alternatively, the opening 34 can be reversibly sealed with a button or zipper mechanism as well as the locking system utilized in certain plastic storage bags such as Zip Lock bags. These examples are non-limiting, and one having skill in the art will understand that there are numerous other ways suitable to provide both a sealable and reversibly sealable opening 34 to envelope 15.

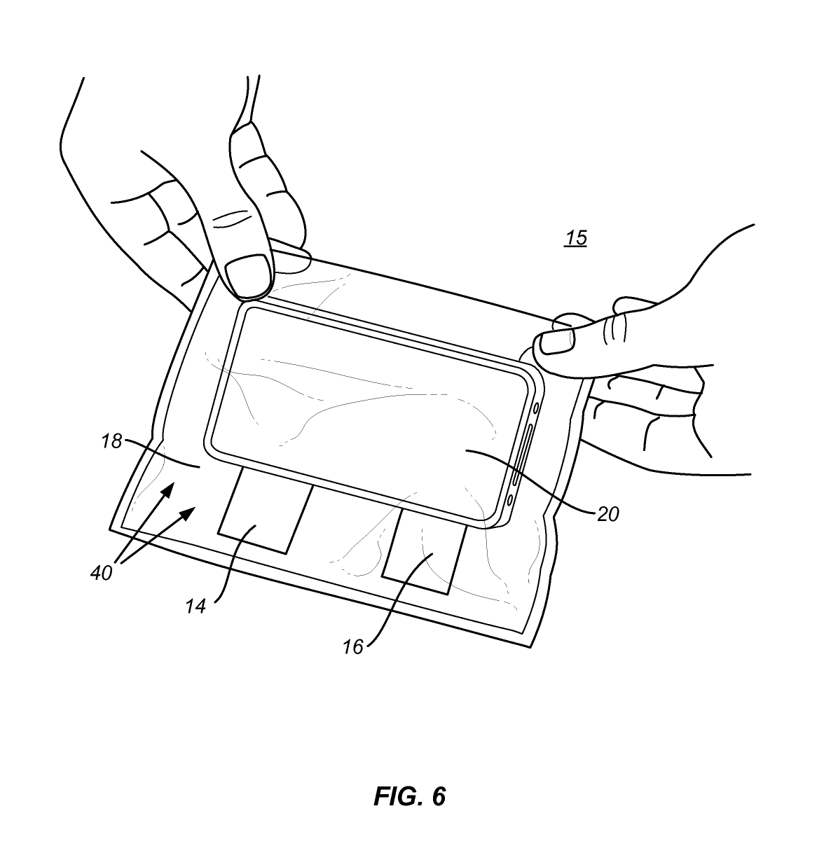

A smartphone with protective cover 20 can be inserted into the bag or envelope 15 as shown in FIG. 6. In this view, the electrically conductive portions 14 and 16 can be seen through the back of the clear envelope 40. The back of the envelope 15 can comprise the insulating portion 18. The ECG sensors are not visible in FIG. 6 because the ECG sensing device is turned away towards the wall of the envelope that comprises the electrically conductive portions 14 and 16. The user placing the ECG sensing device in the envelope 15 can align the sensors of the ECG sensing device so that they communicate with the surface of the electrically conducting portion 14 and 16 of the microbial shield envelope 15 that are on the inside of the microbial shield envelope 15.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An ECG monitoring system comprising
a handheld ECG sensing device comprising two ECG sensors; and
a disposable shield in communication with said two ECG sensors, said disposable shield comprising
a first electrically conductive portion;
a second electrically conductive portion; and
an insulating portion separating said first and said second electrically conducting portions;
wherein said ECG sensing device senses separate electrical potentials on a first and a second skin segment of a subject when said first electrically conductive portion and said second electrically conductive portion are contacted by said first and said second skin segments of said subject.

2. The system of claim 1, wherein said handheld ECG sensing device couples with a smartphone.

3. The system of claim 1, wherein said disposable shield comprises an envelope.

4. The system of claim 3, wherein said envelope comprises a reversibly sealable opening.

5. The system of claim 1, wherein said disposable shield comprises adhesive on at least one surface.

6. The system of claim 1, wherein said first skin segment of said subject comprises a skin segment of a chest of said subject.

7. The system of claim 1, wherein said first skin segment of said subject comprises a skin segment of a limb of said subject.

8. A method for managing contamination of a sensing device, said method comprising
providing a handheld ECG sensing device;
providing a disposable shield;
placing said handheld ECG sensing device in communication with said disposable shield;
placing said disposable shield in contact with a skin surface of a subject, wherein said handheld ECG sensing device senses separate electrical potentials on a first and a second skin segment of a subject without said first and said second skin segments of said subject contacting said handheld ECG sensing device; and
disposing of said disposable shield.

9. The method of claim 8, wherein said handheld ECG sensing device couples with a smartphone.

10. The method of claim 9, wherein said smartphone comprises a display.

11. The method of claim 10, further comprising displaying an ECG of said subject on said display.

12. The method of claim 8, wherein said disposable shield comprises an envelope.

13. The method of claim 12, wherein said envelope comprises a reversibly sealable opening.

14. The method of claim 8, wherein said disposable shield comprises adhesive on at least one surface.

15. The method of claim 8, wherein said first skin segment of said subject comprises a skin segment of a chest of said subject.

16. The method of claim 8, wherein said first skin segment of said subject comprises a skin segment of a limb of said subject.

17. The method of claim 8, wherein said disposable shield comprises a first and a second electrically conductive portion and an insulating portion separating said first and said second electrically conducting portions.
